(19) United States
(12) Patent Application Publication
(10) Pub. No.: US 2005/0119275 A1
(43) Pub. Date: Jun. 2, 2005

(54) Salt and crystal forms of (5-chloro-2-{2-[4-(4-fluoro-benzyl)-(2R,5S)-2,5-dimethyl-piperazin-1-yl]-2-oxo-ethoxy}-phenyl)-methanesulfonic acid

(75) Inventors: Martin A. Berliner, Clinton, CT (US); Matthew M. Hayward, Old Lyme, CT (US); Zheng J. Li, Quaker Hill, CT (US); Clifford N. Meltz, Niantic, CT (US); Karl K. Ng, Warwick, RI (US)

Correspondence Address: PFIZER INC., PATENT DEPARTMENT, MS8260-1611, EASTERN POINT ROAD, GROTON, CT, 06340, US

(73) Assignee: Pfizer Inc.

(21) Appl. No.: 10/871,172

(22) Filed: Jun. 17, 2004

Related U.S. Application Data
(60) Provisional application No. 60/482,525, filed on Jun. 24, 2003.

(51) Int. Cl. A61K 31/495; C07D 241/04

(52) U.S. Cl. 514/255.01; 544/386

(57) ABSTRACT

This invention relates to salt and crystal forms of (5-chloro-2-{2-[4-(4-fluorobenzyl)-(2R,5S)-2,5-dimethyl-piperazin-1-yl]-2-oxo-ethoxy}-phenyl)-methanesulfonic acid, useful in treating or preventing a disorder or condition by antagonizing the CCR1 receptor, and to their methods of preparation and use.

### FIELD OF THE INVENTION

This invention relates to salt and crystal forms of (5-chloro-2-{2-[4-(4-fluoro-benzyl)-(2R,5S)-2,5-dimethyl-piperazin-1-yl]-2-oxo-ethoxy}-phenyl)-methanesulfonic acid and their methods of preparation and use.

### BACKGROUND OF THE INVENTION

(5-Chloro-2-{2-[4-(4-fluoro-benzyl)-(2R,5S)-2,5-dimethyl-piperazin-1-yl]-2-oxo-ethoxy}-phenyl)-methanesulfonic acid (also referred to as “(5-chloro-2-{2-[4-(4-fluoro-benzyl)-2R,5S-dimethyl-piperazin-1-yl]-2-oxo-ethoxy}-phenyl)-methanesufonic acid”) has the chemical formula C₂₂H₂₆ClFN₂O₅S and the following structural formula (Ia):

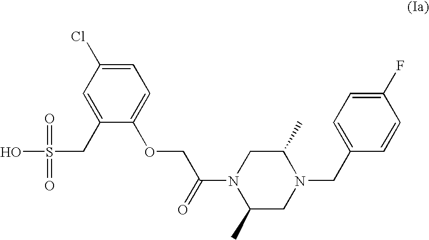

Its synthesis is described in co-pending U.S. patent application Ser. No. 10/175,645, filed Jun. 19, 2002, commonly assigned to the assignee of the present invention which is incorporated herein by reference in its entirety for all purposes.

(5-Chloro-2-{2-[4-(4-fluoro-benzyl)-(2R,5S)-2,5-dimethyl-piperazin-1-yl]-2-oxo-ethoxy}-phenyl)-methanesulfonic acid is a potent inhibitor of MIP-1 a (CCL3) binding to its receptor CCR1 found on inflammatory and immunomodulatory cells (preferably leukocytes and lymphocytes). The CCR1 receptor is also sometimes referred to as the CC-CKR1 receptor. This compound also inhibits MIP-1 a (and the related chemokines shown to interact with CCR1 (e.g., RANTES (CCL5), MCP-2 (CCL8), MCP-3 (CCL7), HCC-1 (CCL14) and HCC-2 (CCL15))) induced chemotaxis of THP-1 cells and human leukocytes and are potentially useful for the treatment and prevention of various disorders and conditions.

### SUMMARY OF THE INVENTION

In one aspect, the present invention relates to crystalline and amorphous forms of (5-chloro-2-{2-[4-(4-fluoro-benzyl)-2R,5S-dimethyl-piperazin-1-yl]-2-oxo-ethoxy}-phenyl)-methanesulfonic acid arginine salt.

In one embodiment, a crystalline form of (5-chloro-2-{2-[4-(4-fluoro-benzyl)-2R,5S-dimethyl-piperazin-1-yl]-2-oxo-ethoxy}-phenyl)-methanesulfonic acid arginine salt form A has a powder X-ray diffraction pattern high intensity peaks expressed in degrees two-theta at approximately 4.1, 12.5, 33.4, 19.5, 20.9 and 24.0; and/or peaks expressed in degrees two-theta at approximately 4.1, 10.8, 11.4, 12.2, 12.5, 12.9, 13.2, 13.7, 14.6, 15.4, 16.1, 16.7, 17.8, 18.2, 18.4, 19.5, 20.1, 20.9, 21.3, 21.8, 22.8, 24.0, 25.1, 25.7, 26.8, 27.1, 28.2, 29.0, 29.5, 30.6, 31.0, and 32.3.

In another embodiment, a crystalline form of (5-chloro-2-{2-[4-(4-fluoro-benzyl)-2R,5S-dimethyl-piperazin-1-yl]-2-oxo-ethoxy}-phenyl)-methanesulfonic acid arginine salt form B has a powder X-ray diffraction pattern comprising peaks expressed in degree two-theta at approximately: 4.0, 11.1, 16.0, 17.3, 17.5, 18.2, 18.4, 19.2, 19.6, 20.0, 21.6, and 22.2.

In another embodiment, a crystalline form of (5-chloro-2-{2-[4-(4-fluoro-benzyl)-2R,5S-dimethyl-piperazin-1-yl]-2-oxo-ethoxy}-phenyl)-methanesulfonic acid arginine salt form C has a powder X-ray diffraction pattern comprising peaks expressed in degree two-theta at approximately: 4.1, 20.5, and 24.7.

In another embodiment, a crystalline form of (5-chloro-2-{2-[4-(4-fluoro-benzyl)-2R,5S-dimethyl-piperazin-1-yl]-2-oxo-ethoxy}-phenyl)-methanesulfonic acid arginine salt form E has a powder X-ray diffraction pattern comprising peaks expressed in degree two-theta at approximately: 3.7, 7.3, 11.0, 18.3, 19.7, 22.1, 22.9, and 25.8.

In another embodiment, the present invention includes a crystalline form of (5-chloro-2-{2-[4-(4-fluoro-benzyl)-2R,5S-dimethyl-piperazin-1-yl]-2-oxo-ethoxy}-phenyl)-methanesulfonic acid arginine salt having a differential scanning calorimetry thermogram comprising an endothermic event with an onset temperature of approximately 200° C. using a heating rate of about 5C per minute from about 30° C. to about 300° C.

A second aspect of the present invention relates to crystalline and amorphous forms of (5-chloro-2-{2-[4-(4-fluoro-benzyl)-2R,5S-dimethyl-piperazin-1-yl]-2-oxo-ethoxy}-phenyl)-methanesulfonic acid ethylene diamine salt.

In one embodiment, a crystalline form of (5-chloro-2-{2-[4-(4-fluoro-benzyl)-2R,5S-dimethyl-piperazin-1-yl]-2-oxo-ethoxy}-phenyl)-methanesulfonic acid ethylene diamine salt has a powder X-ray diffraction pattern comprising high intensity peaks expressed in degrees two-theta at approximately 4.3, 9.5, 18.3, 21.9 and 25.7.

Another embodiment of a crystalline form of (5-chloro-2-{2-[4-(4-fluoro-benzyl)-2R,5S-dimethyl-piperazin-1-yl]-2-oxo-ethoxy}-phenyl)-methanesulfonic acid ethylene diamine salt has a powder X-ray diffraction pattern comprising peaks expressed in degree two-theta at approximately: 4.3, 7.4, 8.3, 8.5, 9.2, 9.5, 11.5, 12.4, 13.2, 14.4, 14.6, 15.0, 16.5, 17.4, 17.6, 18.3, 19.2, 19.5, 20.1, 20.7, 21.0, 21.3, 21.9, 23.4, 23.9, 24.8, 25.7, 27.2, and 28.0.

In another embodiment, a crystalline form of (5-chloro-2-{2-[4-(4-fluoro-benzyl)-2R,5S-dimethyl-piperazin-1-yl]-2-oxo-ethoxy}-phenyl)-methanesulfonic acid ethylene diamine salt has a differential scanning calorimetry thermogram comprising an endothermic event with an onset temperature of approximately 115° C. using a heating rate of about 5C per minute from about 30° C. to about 300° C.

In a third aspect, the present invention relates to amorphous and crystalline forms of (5-chloro-2-{2-[4-(4-fluoro-benzyl)-2R,5S-dimethyl-piperazin-1-yl]-2-oxo-ethoxy}-phenyl)-methanesulfonic acid calcium salt.

In one embodiment, a crystalline form of (5-chloro-2-{2-[4-(4-fluoro-benzyl)-2R,5S-dimethyl-piperazin-1-yl]-2-oxo-ethoxy}-phenyl)-methanesulfonic acid calcium salt has a powder X-ray diffraction pattern comprising high intensity peaks expressed in degrees two-theta at approximately 5.6, 11.4, 12.5 and 22.2.

Another embodiment of a crystalline form of (5-chloro-2-{2-[4-(4-fluoro-benzyl)-2R,5S-dimethyl-piperazin-1-yl]-2-oxo-ethoxy}-phenyl)-methanesulfonic acid calcium salt has a powder X-ray diffraction pattern comprising peaks expressed in degree two-theta at approximately: 3.6, 4.1, 5.6, 6.9, 7.7, 9.5, 11.0, 11.4, 12.5, 14.1, 15.5, 16.5, 17.2, 18.4, 18.9, 19.6, 19.9, 20.4, 21.2, 22.2, 22.9, 23.4, 23.8, 24.2, 25.1, 25.2, 26.2, 27.0, 27.8, 28.2, 28.7, 29.6, 30.1, 31.0, 32.5, 33.6, 34.1, 34.8, 35.8, 36.6, 37.1, 37.7, 37.9, 38.3, 39.0, and 39.5.

In another embodiment, a crystalline form of (5-chloro-2-{2-[4-(4-fluoro-benzyl)-2R,5S-dimethyl-piperazin-1-yl]-2-oxo-ethoxy}-phenyl)-methanesulfonic acid calcium salt has a differential scanning calorimetry thermogram comprising an endothermic event with an onset temperature of approximately 120° C. using a heating rate of about 5° C. per minute from about 30° C. to about 300° C.

In another embodiment, a crystal form of (5-chloro-2-{2-[4-(4-fluoro-benzyl)-2R,5S-dimethyl-piperazin-1-yl]-2-oxo-ethoxy}-phenyl)-methanesulfonic acid arginine salt form A has a solid state nuclear magnetic resonance spectrum comprising ¹³c chemical shifts expressed in parts per million at approximately 174.9, 174.1, 167.3, 166.4, 163.3, 162.8, 161.4, 160.8, 158.8, 157.2, 154.4, 134.3, 133.7, 132.3, 131.5, 130.8, 128.1, 125.8, 124.7, 123.6, 117.7, 116.8, 114.7, 111.7, 72.6, 67.0, 58.1, 56.0, 55.1, 52.8, 51.9, 51.0, 49.0, 46.8, 43.0, 41.3, 27.7, 26.3, 24.8, 23.3, 17.9, 15.4, 9.5, and 7.4.

In a fourth aspect, the present invention relates to pharmaceutical compositions comprising an amount of a compound of described above, or a pharmaceutically acceptable salt thereof, and a pharmaceutically acceptable carrier.

In a fifth aspect, the present invention relates to methods for treating or preventing a disorder or condition that can be treated or prevented by antagonizing the CCR1 receptor in a subject or inhibiting the production of metalloproteinase or cytokine at an inflammatory site in a subject, wherein the method comprises administering to said subject an effective amount of the compounds or compositions described above.

As used herein, the term “effective amount” refers to an amount of a compound or composition of the invention required to treat or prevent a disorder or condition in a subject that can be treated or prevented by antagonizing the CCR1 receptor or inhibiting the production of metalloproteinase or cytokine at an inflammatory site. As would be understood by one of skill in the art, an “effective amount will vary from subject to subject and will be determined on a case by case basis. Factors to consider include, but are not limited to, the subject being treated, weight, health, compound administered, the severity of the disorder or condition, the rate of administration and the judgment of the prescribing physician, etc.

In one embodiment, the methods are useful for treating or preventing a disorder or condition selected from the group consisting of autoimmune diseases, acute and chronic inflammatory conditions, allergic conditions, infection associated with inflammation, viral inflammation, transplantation tissue rejection, atherosclerosis, restenosis, HIV infectivity, granulomatous diseases in a mammal, fibrosis, Alzheimer's disease, conditions associated with leptin production, sequelae associated with cancer, cancer metastasis, diseases or conditions related to production of cytokines at inflammatory sites, and tissue damage caused by inflammation induced by infectious agents.

In another embodiment, the methods are useful for treating or preventing disorders or conditions selected from the group consisting of rheumatoid arthritis, Takayasu arthritis, psoriatic arthritis, ankylosing spondylitis, type I diabetes (recent onset), lupus, inflammatory bowel disease, Chrohn's disease, optic neuritis, psoriasis, multiple sclerosis, polymyalgia rheumatica, uveitis, thyroiditis and vasculitis, pulmonary fibrosis, fibrosis associated with end-stage renal disease, fibrosis caused by radiation, tubulointerstitial fibrosis, subepithelial fibrosis, scleroderma, hepatic fibrosis, primary and secondary biliary cirrhosis, asthma, contact dermatitis, atopic dermatitis, chronic bronchitis, chronic obstructive pulmonary disease, adult Respiratory Distress Syndrome, Respiratory Distress Syndrome of infancy, immune complex alveolitis, synovial inflammation caused by arthroscopy, hyperuremia, osteoarthritis, ischemia reperfusion injury, glomerulonephritis, nasal polyosis, enteritis, Behcet's disease, preeclampsia, oral lichen planus, Guillian-Barre syndrome, sarcoidosis, leprosy, tuberculosis, obesity, cachexia, anorexia, type II diabetes, hyperlipidemia and hypergonadism, sequelae associated with multiple myeloma, breast cancer, joint tissue damage, hyperplasia, pannus formation and bone resorption, hepatic failure, Kawasaki syndrome, myocardial infarction, acute liver failure, septic shock, congestive heart failure, pulmonary emphysema or dyspnea associated therewith, viral induced encephalomyelitis or demyelination, viral inflammation of the lung or liver, gastrointestinal inflammation, bacterial meningitis, cytomegalovirus, adenoviruses, Herpes viruses, fungal meningitis, lyme disease, and malaria.

It is to be understood that both the foregoing general description and the following detailed description are exemplary and explanatory only and are not restrictive of the invention, as claimed.

### BRIEF DESCRIPTION OF THE DRAWINGS

FIG. 1 is a representative powder X-ray diffraction pattern for (5-Chloro-2-{2-[4-(4-fluoro-benzyl)-2R,5S-dimethyl-piperazin-1-yl]-2-oxo-ethoxy}-phenyl)-methanesulfonic acid arginine salt, form A, (Vertical Axis: Intensity (counts); Horizontal Axis: Two Theta (Degrees)).

FIG. 2 is a representative powder X-ray diffraction pattern for (5-Chloro-2-{2-[4-(4-fluoro-benzyl)-2R,5S-dimethyl-piperazin-1-yl]-2-oxo-ethoxy}-phenyl)-methanesulfonic acid arginine salt, form B, (Vertical Axis: Intensity (counts); Horizontal Axis: Two Theta (Degrees)).

FIG. 3 is a representative powder X-ray diffraction pattern for (5-Chloro-2-{2-[4-(4-fluoro-benzyl)-2R,5S-dimethyl-piperazin-1-yl]-2-oxo-ethoxy}-phenyl)-methanesulfonic acid arginine salt, form C, (Vertical Axis: Intensity (counts); Horizontal Axis: Two Theta (Degrees)).

FIG. 4 is a representative powder X-ray diffraction pattern for (5-Chloro-2-{2-[4-(4-fluoro-benzyl)-2R,5S-dimethyl-piperazin-1-yl]-2-xo-ethoxy}-phenyl)-methanesulfonic acid arginine salt, form E, (Vertical Axis: Intensity (counts); Horizontal Axis: Two Theta (Degrees)).

FIG. 5 is a representative differential scanning calorimetry thermogram of (5-Chloro-2-{2-[4-(4-fluoro-benzyl)-2R,5S-dimethyl-piperazin-1-yl]-2-oxo-ethoxy}-phenyl)-methanesulfonic acid arginine salt, form A, (Scan Rate: 5° C. per minute; Vertical Axis: Heat Flow (w/g); Horizontal Axis: Temperature (° C.)).

FIG. 6 is a representative ¹³C solid state nuclear magnetic resonance spectrum for (5-Chloro-2-{2-[4-(4-fluoro-benzyl)-2R,5S-dimethyl-piperazin-1-yl]-2-oxo-ethoxy}-phenyl)-methanesulfonic acid arginine salt, form A, (Vertical Axis: Intensity (counts); Horizontal Axis: Chemical shift (6-scale), in ppm).

FIG. 7 is a representative powder X-ray diffraction pattern for (5-Chloro-2-{2-[4-(4-fluoro-benzyl)-2R,5S-dimethyl-piperazin-1-yl]-2-oxo-ethoxy}-phenyl)-methanesulfonic acid ethylene diamine salt. (Vertical Axis: Intensity (counts); Horizontal Axis: Two Theta (Degrees)).

FIG. 8 is a representative differential scanning calorimetry thermogram of (5-Chloro-2-{2-[4-(4-fluoro-benzyl)-2 R,5S-dimethyl-piperazin-1-yl]-2-oxo-ethoxy}-phenyl)-methanesulfonic acid ethylene diamine salt. (Scan Rate: 5° C. per minute; Vertical Axis: Heat Flow (w/g); Horizontal Axis: Temperature (° C.)). Presence of acetonitrile contributes to first event (from about 32° C. to about 58° C.).

FIG. 9 is a representative powder X-ray diffraction pattern for (5-Chloro-2-{2-[4-(4-fluoro-benzyl)-2R,5S-dimethyl-piperazin-1-yl]-2-oxo-ethoxy}-phenyl)-methanesulfonic acid calcium salt. (Vertical Axis: Intensity (counts); Horizontal Axis: Two Theta (Degrees)).

FIG. 10 is a representative differential scanning calorimetry thermogram of (5-Chloro-2-{2-[4-(4-fluoro-benzyl)-2R,5S-dimethyl-piperazin-1-yl]-2-oxo-ethoxy}-phenyl)-methanesulfonic acid calcium salt. (Scan Rate: 5° C. per minute; Vertical Axis: Heat Flow (w/g); Horizontal Axis: Temperature (° C.)). Presence of water contributes to first event (from about 88° C. to about 102° C.).

### DETAILED DESCRIPTION OF THE INVENTION

The present invention may be understood more readily by reference to the following detailed description of exemplary embodiments of the invention and the examples included therein.

Before the present salt and crystal forms and methods are disclosed and described, it is to be understood that this invention is not limited to specific synthetic methods of making that may of course vary. It is also to be understood that the terminology used herein is for the purpose of describing particular embodiments only and is not intended to be limiting.

In this specification and in the claims that follow, reference will be made to a number of terms that shall be defined to have the following meanings:

By “pharmaceutically acceptable” is meant a material that is not biologically or otherwise undesirable, i.e., the material may be administered to an individual along with the selected compound without causing any undesirable biological effects or interacting in a deleterious manner with any of the other components of the pharmaceutical composition in which it is contained.

The term “subject” is meant an individual. Preferably, the subject is a mammal such as a primate, and more preferably, a human. Thus, the “subject” can include domesticated animals (e.g., cats, dogs, etc.), livestock (e.g., cattle, horses, pigs, sheep, goats, etc.), and laboratory animals (e.g., mouse, rabbit, rat, guinea pig, etc.).

In general, “effective amount” or “effective dose” means the amount needed to achieve the desired result or results (treating or preventing the condition). One of ordinary skill in the art will recognize that the potency and, therefore, an “effective amount” can vary for the various compounds used in the invention. One skilled in the art can readily assess the potency of the compounds.

The salt forms of the present invention may be referred to herein by their salt. As such, “the arginine salt” refers to 5-chloro-2-{2-[4-(4-fluoro-benzyl)-(2R,5S)-2,5-dimethyl-piperazin-1-yl]-2-oxo-ethoxy}-phenyl)-methanesulfonic acid arginine salt and “the crystalline arginine salt” refers to all crystalline forms of 5-chloro-2-{2-[4-(4-fluoro-benzyl)-(2R,5S)-2,5-dimethyl-piperazin-1-yl]-2-oxo-ethoxy}-phenyl)-methanesulfonic acid arginine salt. Similarly, “the ethylene diamine salt” refers to (5-chloro-2-{2-[4-(4-fluoro-benzyl)-(2R,5S)-2,5-dimethyl-piperazin-1-yl]-2-oxo-ethoxy}-phenyl)-methanesulfonic acid ethylene diamine salt and “the crystalline ethylene diamine salt” refers to all crystalline forms of (5-chloro-2-{2-[4-(4-fluoro-benzyl)-(2R,5S)-2,5-dimethyl-piperazin-1-yl]-2-oxo-ethoxy}-phenyl)-methanesulfonic acid ethylene diamine salt; and “the calcium salt” refers to (5-chloro-2-{2-[4-(4-fluoro-benzyl)-(2R,5S)-2,5-dimethyl-piperazin-1-yl]-2-oxo-ethoxy}-phenyl)-methanesulfonic acid calcium salt” and “the crystalline calcium salt” refers to all crystalline forms of the (5-chloro-2-{2-[4-(4-fluoro-benzyl)-(2R,5S)-2,5-dimethyl-piperazin-1-yl]-2-oxo-ethoxy}-phenyl)-methanesulfonic acid calcium salt.

Unless otherwise noted, numerical values described and claimed herein are approximate. Variation within the values may be attributed to equipment calibration, equipment errors, purity of the materials, crystal size, and sample size, among other factors. Additionally, variation may be possible, while still obtaining the same result. For example, X-ray diffraction values are generally accurate to within +0.2 2-theta degrees, preferably to within ±0.2 2-theta degrees. Similarly, DSC results are typically accurate to within about 2° C., preferably to within 1.5° C.

The crystalline state of a compound can be described by several crystallographic parameters including single crystal structure and powder crystal X-ray diffraction pattern. Such crystalline description is advantageous because a compound may have more than one type of crystal form. It has been discovered that there are several crystal forms of (5-Chloro-2-{2-[4-(4-fluoro-benzyl)-2R,5S-dimethyl-piperazin-1-yl]-2-oxo-ethoxy}-phenyl)-methanesulfonic acid for the arginine salt, ethylene diamine salt, and calcium salt.

To describe and distinguish the crystal forms, the crystalline salts have each been examined by powder X-ray diffraction and differential scanning calorimetry (DSC). A discussion of the theory of X-ray power diffraction patterns can be found in Stout & Jensen, X-Ray Structure Determination: A Practical Guide, MacMillan Co., New York, N.Y. (1968), which is incorporated by reference in its entirety for all purposes.

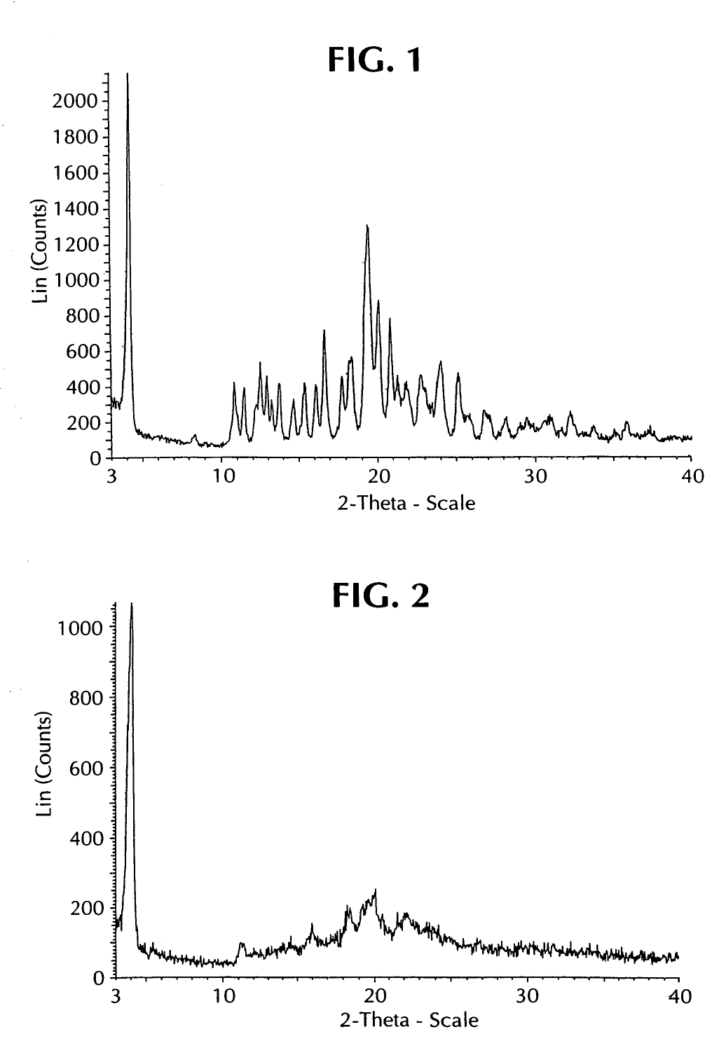

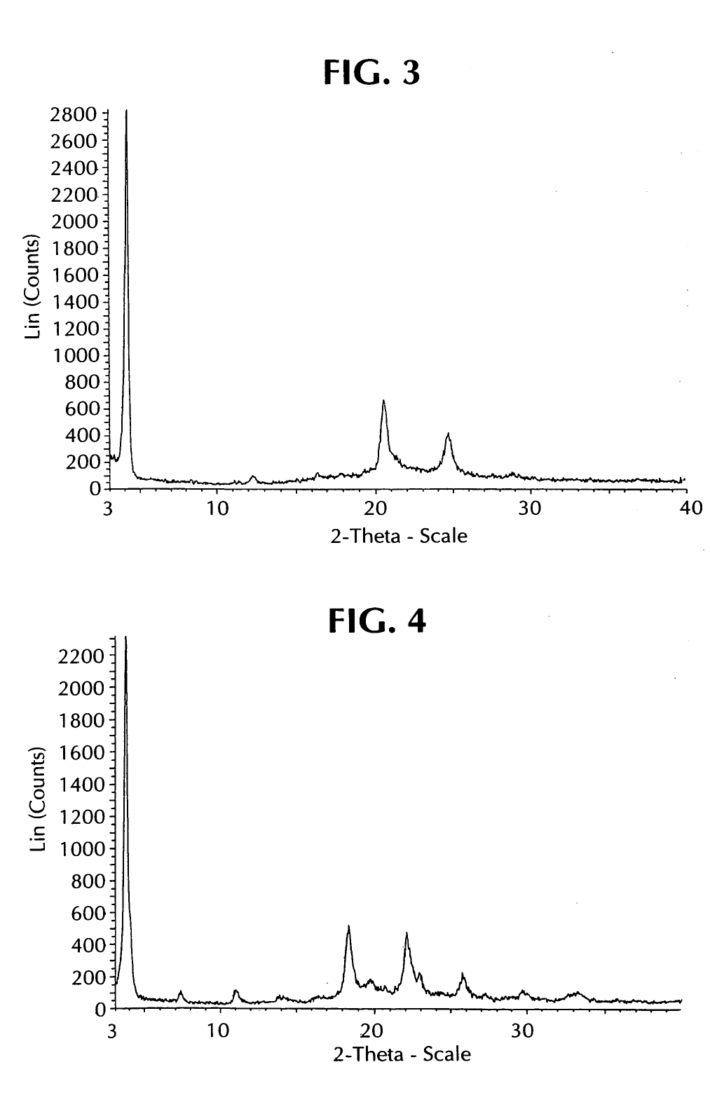

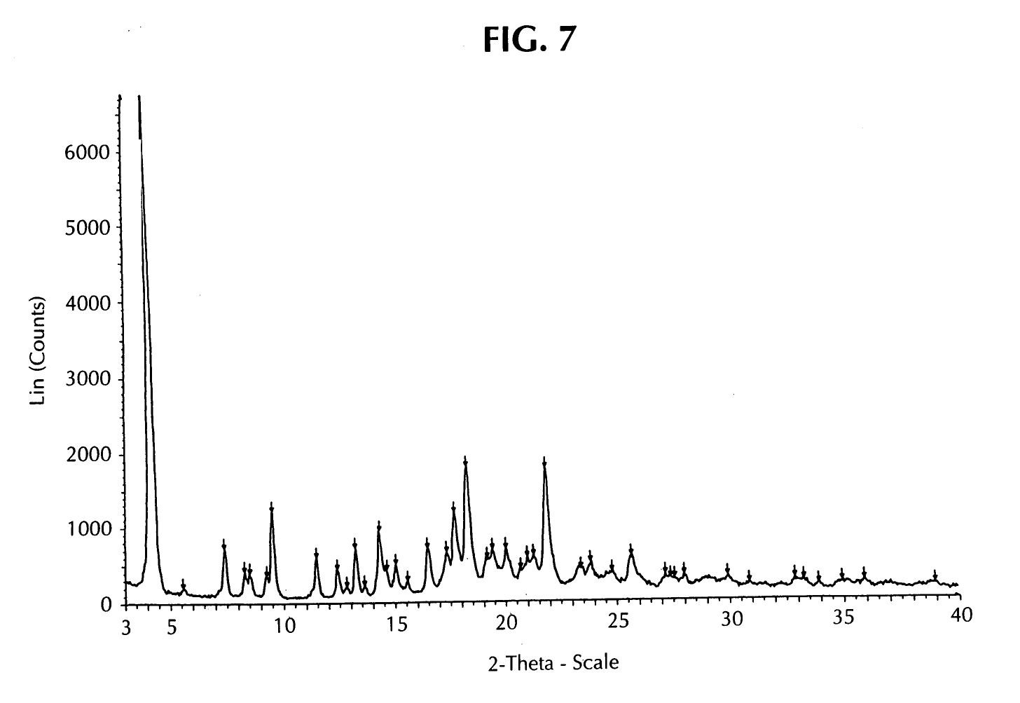

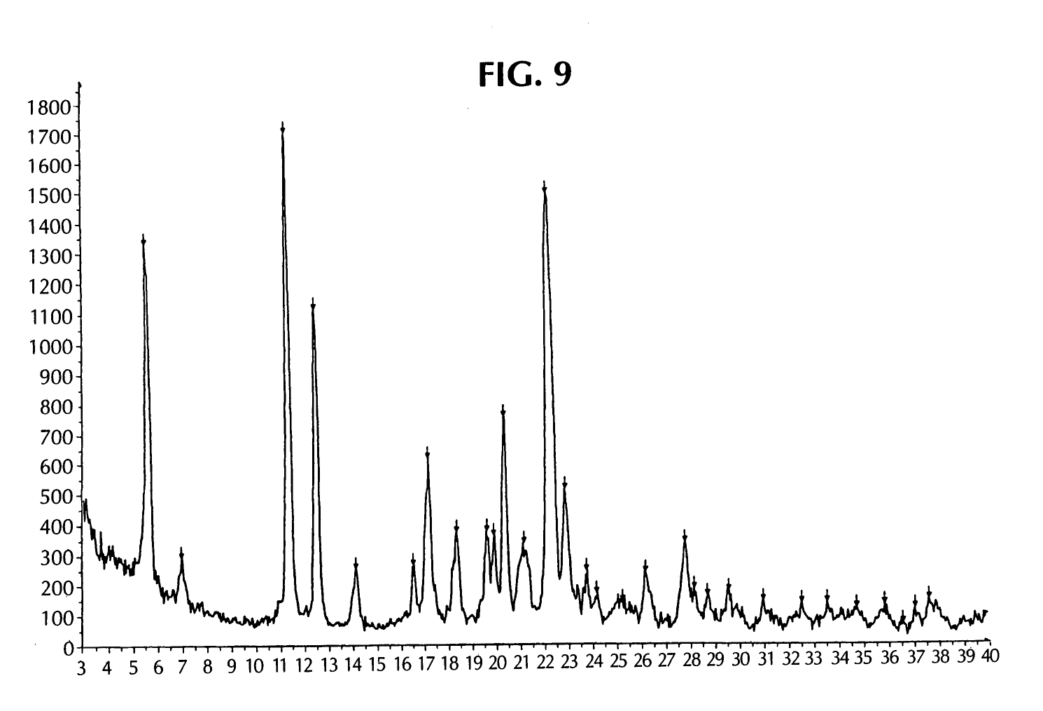

Crystallographic data on a collection of powder crystals provides powder X-ray diffraction. The crystalline salt forms of the present invention have distinctive powder X-ray diffraction patterns. The powder X-ray diffraction patterns of the represented crystalline arginine salt forms A, B, C and E, crystalline ethylene diamine salt, and crystalline calcium salt are depicted, respectively, in FIGS. 1, 2, 3, 4, 7, and 9. The experimental conditions under which the powder X-ray diffraction was conducted are as follows: Cu anode; wavelength 1: 1.54056; wavelength 2: 1.54439 (Relative Intensity: 0.500); range # 1—coupled: 3.000 to 40.000; step size: 0.040; step time: 1.00; smoothing width: 0.300; and threshold: 1.0.

The powder X-ray diffraction patterns display high intensity peaks, which are useful in identifying a specific crystal form. However, the relative intensities are dependent upon several factors, including, but not limited to, crystal size and morphology. As such, the relative intensity values may very from sample to sample. The powder X-ray diffraction values are generally accurate to within ±0.2 2-theta degrees, due to slight variations of instrument and test conditions. The powder X-ray diffraction patterns or a collective of the diffraction peaks for each of the salts provide a qualitative test for comparison against uncharacterized crystals. The diffraction peaks detected with greater than 5% relative intensity are provided in Tables 1-6.

TABLE 1
Powder X-ray Diffraction Peaks-
Represented Crystalline Arginine Salt Form A

| Angle | Rel. Intensity |
|---|---|
| 2-Theta ° | % |
| 4.1 | 100 |
| 10.8 | 19.6 |
| 11.4 | 18.3 |
| 12.2 | 13.8 |
| 12.5 | 25 |
| 12.9 | 21.4 |
| 13.2 | 15.1 |
| 13.7 | 19.4 |
| 14.6 | 15.2 |
| 15.4 | 19.6 |
| 16.1 | 19.1 |
| 16.7 | 33.4 |
| 17.8 | 21.2 |
| 18.2 | 16 |
| 18.4 | 26.3 |
| 19.5 | 61 |
| 20.1 | 41.1 |
| 20.9 | 36.6 |
| 21.3 | 21.5 |
| 21.8 | 19.9 |
| 22.8 | 21.6 |
| 24.0 | 25.3 |
| 25.1 | 22.2 |
| 25.7 | 10.7 |
| 26.8 | 12.2 |
| 27.1 | 11.1 |
| 28.2 | 9.9 |
| 29.0 | 7.7 |
| 29.5 | 10.3 |
| 30.6 | 9.5 |
| 31.0 | 10.3 |
| 32.3 | 12 |

TABLE 2
Powder X-ray Diffraction Peaks-
Represented Crystalline Arginine Salt Form B

| Angle | Rel. Intensity |
|---|---|
| 2-Theta ° | % |
| 3.967 | 100 |
| 11.107 | 9.3 |
| 15.985 | 14.4 |
| 17.284 | 9.7 |
| 17.51 | 11.6 |
| 18.2 | 19.3 |
| 18.44 | 18.5 |
| 19.201 | 19.6 |
| 19.607 | 21 |
| 20.036 | 23.8 |
| 21.572 | 17.4 |
| 22.2 | 17.4 |

TABLE 3
Powder X-ray Diffraction Peaks-
Represented Crystalline Arginine Salt Form C

| Angle | Rel. Intensity |
|---|---|
| 2-Theta ° | % |
| 4.1 | 100 |
| 20.541 | 23.4 |
| 24.656 | 15 |

TABLE 4
Powder X-ray Diffraction Peaks-
Represented Crystalline Arginine Salt Form E

| Angle | Rel. Intensity |
|---|---|
| 2-Theta ° | % |
| 3.709 | 100 |
| 7.307 | 4.7 |
| 11.039 | 4.9 |
| 18.366 | 22.4 |
| 19.745 | 8.1 |
| 22.09 | 20.6 |
| 22.897 | 9.7 |
| 25.825 | 8.5 |

TABLE 5
Powder X-ray Diffraction Peaks- Represented
Crystalline Ethylene Diamine Salt

| | Rel. |
|---|---|
| Angle | Intensity |
| 2-theta° | (%) |
| 4.3 | 100 |
| 7.4 | 10.7 |
| 8.3 | 6.1 |
| 8.5 | 5.8 |
| 9.2 | 5.3 |
| 9.5 | 18.1 |
| 11.5 | 9.0 |
| 12.4 | 6.6 |
| 13.2 | 10.8 |
| 14.4 | 14.2 |
| 14.6 | 6.5 |
| 15.0 | 7.6 |
| 16.5 | 10.7 |
| 17.4 | 10.0 |
| 17.8 | 17.8 |
| 18.3 | 26.9 |
| 19.2 | 8.8 |
| 19.5 | 10.6 |
| 20.1 | 10.6 |
| 20.7 | 6.5 |
| 21.0 | 8.9 |
| 21.3 | 9.3 |
| 21.9 | 26.6 |
| 23.4 | 6.5 |
| 23.9 | 7.9 |
| 24.8 | 5.8 |
| 25.7 | 9.1 |
| 27.2 | 5.2 |
| 28.0 | 5.0 |

TABLE 6
Powder X-ray Diffraction Peaks- ReDresented Crystalline Calcium Salt

| | Rel. |
|---|---|
| Angle | Intensity |
| 2-theta° | (%) |
| 3.6 | 22.2 |
| 4.1 | 18.6 |
| 5.6 | 77.8 |
| 6.9 | 17.1 |
| 7.7 | 8.6 |
| 9.5 | 5.9 |
| 11.0 | 9.0 |
| 11.4 | 100 |
| 12.5 | 65.7 |
| 14.1 | 15.3 |
| 15.5 | 5.4 |
| 16.5 | 16.1 |
| 17.2 | 36.9 |
| 18.4 | 22.6 |
| 18.9 | 6.2 |
| 19.6 | 22.8 |
| 19.9 | 22.0 |
| 20.4 | 45.2 |
| 21.2 | 20.6 |
| 22.2 | 88.6 |
| 22.9 | 31.0 |
| 23.4 | 12.2 |
| 23.8 | 15.3 |
| 24.2 | 10.8 |
| 25.1 | 9.5 |
| 25.2 | 9.1 |
| 26.2 | 15.1 |
| 27.0 | 6.4 |
| 27.8 | 21.0 |
| 28.2 | 11.9 |
| 28.7 | 10.4 |
| 29.6 | 11.5 |
| 30.1 | 8.0 |
| 31.0 | 9.6 |
| 32.5 | 9.1 |
| 33.6 | 9.3 |
| 34.1 | 8.0 |
| 34.8 | 8.3 |
| 35.8 | 9.0 |
| 36.6 | 5.2 |
| 37.1 | 8.3 |
| 37.7 | 10.1 |
| 37.9 | 9.6 |
| 38.3 | 6.0 |
| 39.0 | 6.3 |
| 39.5 | 7.2 |

Moreover, each represented crystalline salt form has high intensity peaks at two-theta:
Crystalline Arginine salt form A: 4.1, 12.5, 16.7, 18.4, 19.5, 20.1, 20.9 and 24.0
Crystalline Ethylene diamine salt: 4.3, 9.5, 18.3, 21.9 and 25.7
Crystalline Calcium salt: 5.6, 11.4, 12.5 and 22.2

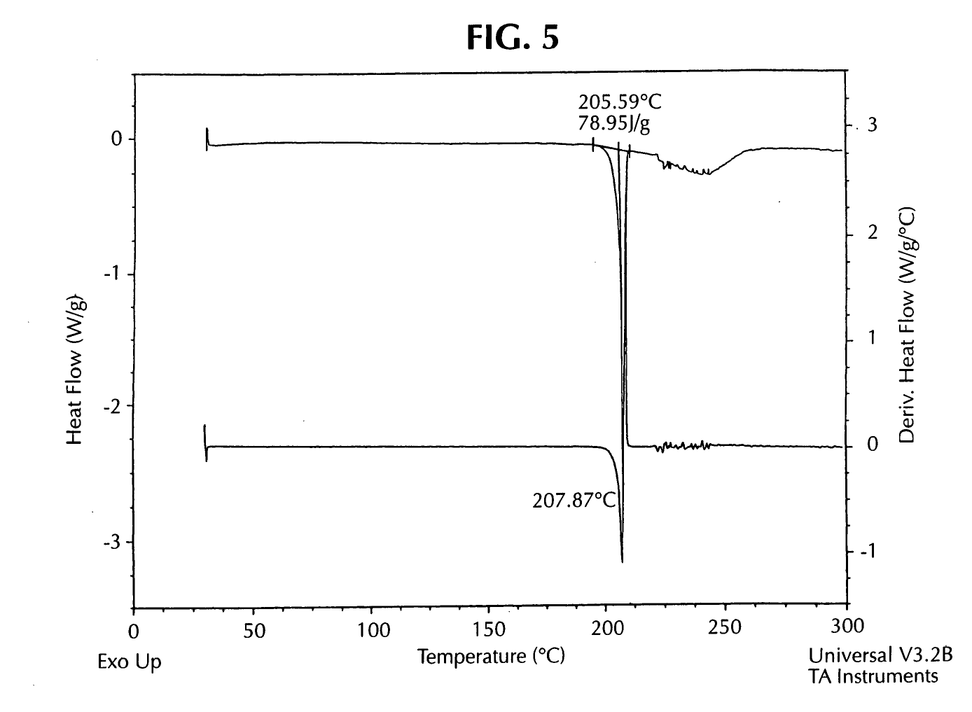

Differential Scanning Calorimetry (DSC) analysis was carried out on either TA Instruments DSC2920 or a Mettler DSC 821, calibrated with indium. DSC samples were prepared by weighing 24 mg of material in an aluminum pan with a pinhole. The sample was heated under nitrogen, at a rate of 5° C. per minute from about 30° C. to about 300° C. The onset temperature of the melting endotherm was reported as the melting temperature. The differential scanning calorimetry (DSC) thermograms for the represented crystalline arginine salt, crystalline ethylene diamine salt, and crystalline calcium salt forms are shown, respectively, in FIGS. 5, 8, and 10. The onset temperature of the melting endotherm is dependent on the rate of heating, the purity of the sample, crystal size and sample size, among other factors. Typically, the DSC results are accurate to within about ±2° C., preferably to within ±1.5° C. The thermograms may be interpreted as follows.

Referring to FIG. 5, the represented crystalline arginine salt form A exhibits an endotherm with an onset temperature of about 200° C.

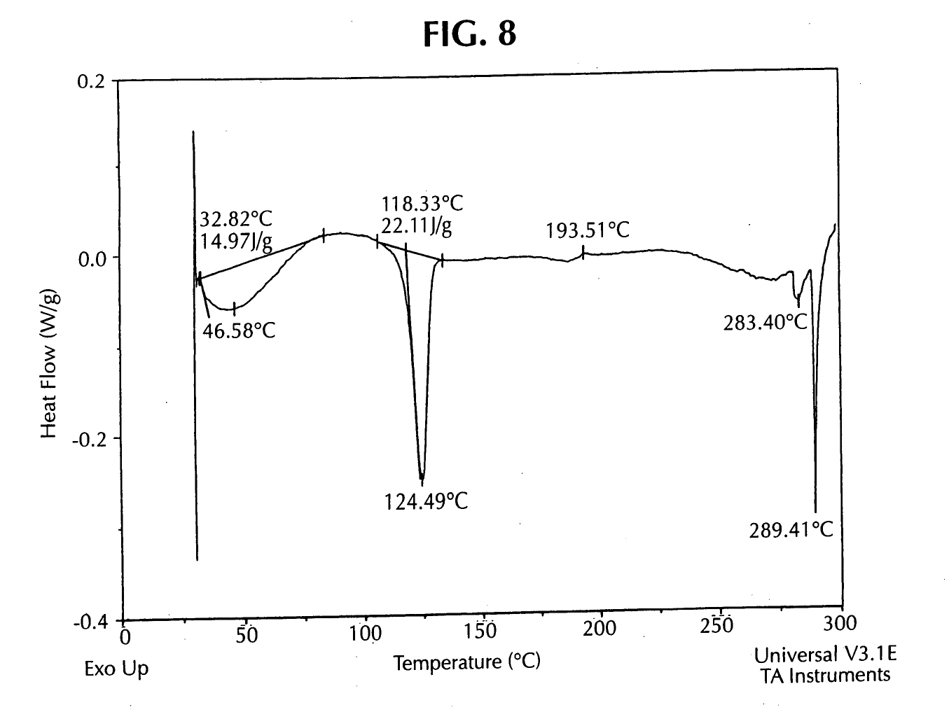

Referring to FIG. 8, the represented crystalline ethylene diamine salt exhibits an endotherm with an onset temperature of about 115° C.

Referring to FIG. 10, the represented crystalline calcium salt exhibits an endotherm with an onset temperature of about 120° C.

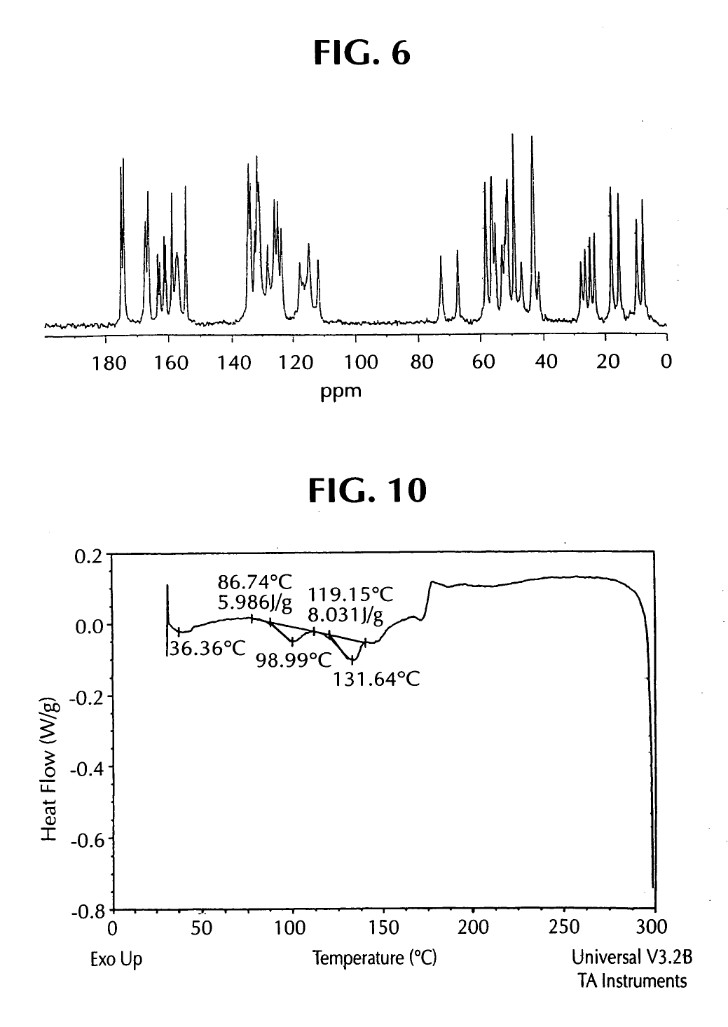

¹³C solid state nuclear magnetic resonance (ss-NMR) provides unique ¹³C chemical shifts spectra for each crystal form. (5-Chloro-2-{2-[4-(4-fluorobenzyl)-2R,5S-dimethyl-piperazin-1-yl]-2-oxo-ethoxy}-phenyl)-methanesulfonic acid arginine salt, form A has been analyzed with ss-NMR and is depicted in FIG. 6. The experimental conditions under which the ss-NMR was conducted are as follows: collected on 11.75 T spectrometer (Bruker Biospin, Inc., Billerica, Mass.), corresponding to 125 MHz 13C frequency and acquired using cross-polarization magic angle spinning (CPMAS) probe operating at ambient temperature and pressure. 4 mm BL Bruker probes were employed, accommodating 75 mg of sample with maximum speed of 15 kHz. Data were processed with exponential line broadening function of 5.0 Hz. Proton decoupling of 100 kHz was used. Sufficient number of acquisitions were averaged out to obtain adequate signal-to-noise ratios for all peaks. Typically, 1500 scans were acquired with recycle delay of 4.5 s, corresponding to approximately 2-hour total acquisition time. Magic angle was adjusted using KBr powder according to standard NMR vendor practices. The spectra were referenced relative to the up-field resonance of adamantane (ADMNT) at 29.5 ppm. The spectral window minimally included the spectra region from 220 to −10 ppm. ¹³C chemical shifts between about 0 to 50 ppm and about 110 to 180 ppm may be useful in identifying the crystal form. The chemical shift data is dependent on the testing conditions (i.e. spinning speed and sample holder), reference material, and data processing parameters, among other factors. Typically, the ss-NMR results are accurate to within about ±0.2 ppm.

The ¹³C chemical shifts of (5-Chloro-2-{2-[4-(4-fluoro-benzyl)-2R,5S-dimethyl-piperazin-1-yl]-2-oxo-ethoxy}-phenyl)-methanesulfonic acid arginine salt, form A is shown in Table 7.

TABLE 7
¹³C ss_NMR Chemical Shifts for (5-Chloro-2-{2-[4-(4-fluoro-
benzyl)-2R,5S-dimethyl-piperazin-1-yl]-2-oxo-ethoxy}-phenyl)-
methanesulfonic acid arginine salt, form A

174.9
174.1
167.3
166.4
163.3
162.8
161.4
160.8
158.8
157.2
154.4
134.3
133.7
132.3
131.5
130.8
128.1
125.8
124.7
123.6
117.7
116.8
114.7
111.7
72.6
67.0
58.1
56.0
55.1
52.8
51.9
51.0
49.0
46.8
43.0
41.3
27.7
26.3
24.8
23.3
17.9
15.4
9.5
7.4

The salt forms of the compound of this invention (5-chloro-2-{2-[4-(4-fluoro-benzyl)-(2R,5S)-2,5-dimethyl-piperazin-1-yl]-2-oxo-ethoxy}-phenyl)-methanesulfonic acid arginine salt, (5-chloro-2-{2-[4-(4-fluoro-benzyl)-(2R,5S)-2,5-dimethyl-piperazin-1-yl]-2-oxo-ethoxy}-phenyl)-methanesulfonic acid ethylene diamine salt and (5-chloro-2-{2-[4-(4-fluoro-benzyl)-(2R,5S)-2,5-dimethyl-piperazin-1-yl]-2-oxo-ethoxy}-phenyl)-methanesulfonic acid calcium salt may be prepared using any suitable method including the method according to Scheme 1.

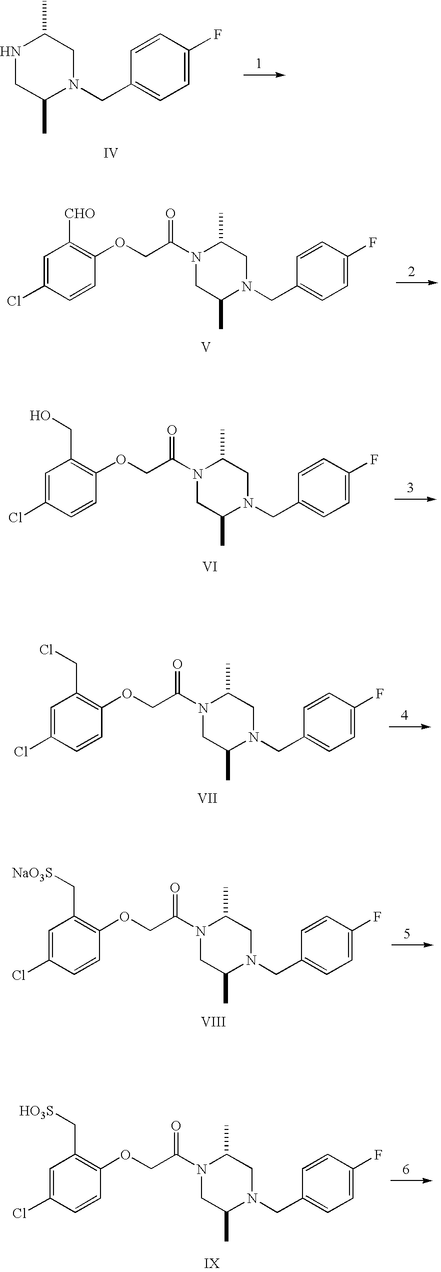

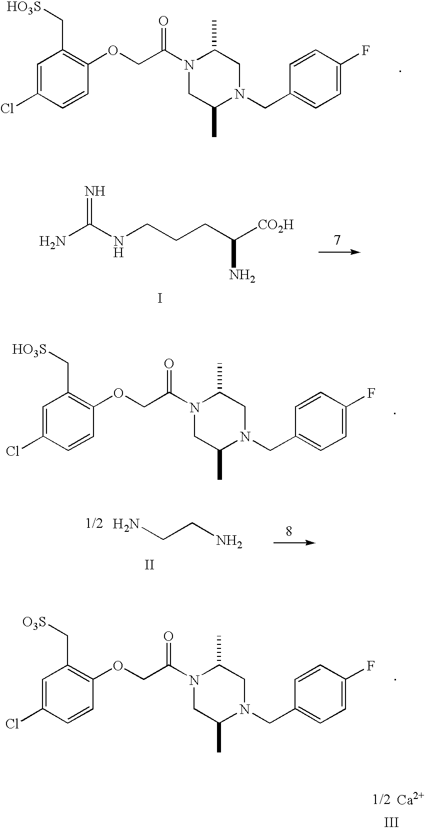

Compounds of formula IV may be produced by any process, including the processes described in U.S. patent application Ser. No. 10/175,645, filed Jun. 19, 2002, which is incorporated herein by reference in its entirety for all purposes.

In reaction 1 the 1-(4-fluoro-benzyl)-2R,5S-dimethyl-piperazine of formula IV is converted to the corresponding 5-chloro-2-{2-[4-(4-fluoro-benzyl)-2R,5S-dimethyl-piperazin-1-yl]-2-oxo-ethoxy}-benzaldehyde of formula V by first reacting IV with chloroacetyl chloride in the presence of a base, such as triethylamine, in an aprotic solvent, such as toluene. The reaction is stirred for a time period of 30 minutes to about 4 hours, preferably 1 hour.

The resulting acetyl chloride is reacted with 5-chlorosalicylaldehyde in the presence of a base, such as potassium carbonate, in an aprotic solvent, such as toluene or dimethylformamide, at a temperature between 60° C. and 100° C., preferably 85° C. The reaction is stirred for a time period of 2 to 20 hours, preferably 12 hours.

In reaction 2 the 5-chloro-2-{2-[4-(4-fluoro-benzyl)-2R,5S-dimethyl-piperazin-1-yl]-2-oxo-ethoxy}-benzaldehyde of formula V is converted to the corresponding 2-(4-chloro-2-hydroxymethyl-phenoxy)-1-[4-(4-fluoro-benzyl)-2R,5S-dimethyl-piperazin-1-yl]-ethanone of formula VI by reacting V with a reducing agent, such as sodium borohydride, in a solvent such as ethanol or tetrahydrofuran. The reaction is stirred for a time period of 30 minutes to about 2 hours, preferably 1 hour.

In reaction 3 the 2-(4-chloro-2-hydroxymethyl-phenoxy)-1-[4-(4-fluoro-benzyl)-2R,5S-dimethyl-piperazin-1-yl]-ethanone of formula VI is converted to the corresponding 2-(4-chloro-2-chloromethyl-phenoxy)-1-[4-(4-fluoro-benzyl)-2R,5S-dimethyl-piperazin-1-yl]-ethanone of formula VII by reacting VI with thionyl chloride in an aprotic solvent such as methylene chloride. The reaction is stirred for a time period of 30 minutes to about 2 hours, preferably 1 hour.

In reaction 4 the 2-(4-chloro-2-chloromethyl-phenoxy)-1-[4-(4-fluoro-benzyl)-2R,5S-dimethyl-piperazin-1-yl]-ethanone of formula VII is converted to the corresponding (5-chloro-2-{2-[4-(4-fluoro-benzyl)-2R,5S-dimethyl-piperazin-1-yl]-2-oxo-ethoxy}-phenyl)-methanesulfonic acid sodium salt of formula Vil by reacting VII with sodium bisulfite in solvent mixtures containing amounts of ethanol, acetonitrile or water, at a temperature between 60° C. and 100° C., preferably about 85° C. The reaction is stirred for a time period of 2 to 12 hours, preferably 6 hours.

In reaction 5 the (5-chloro-2-{2-[4-(4-fluoro-benzyl)-2R,5S-dimethyl-piperazin-1-yl]-2-oxo-ethoxy}-phenyl)-methanesulfonic acid sodium salt of formula VII is converted to the corresponding (5-chloro-2-{2-[4-(4-fluoro-benzyl)-2R,5S-dimethyl-piperazin-1-yl]-2-oxo-ethoxy}-phenyl)-methanesulfonic acid of formula IX by reaction Vil with hydrochloric acid in a solvent, such as acetonitrile.

In reaction 6 the (5-chloro-2-{2-[4-(4-fluoro-benzyl)-2R,5S-dimethyl-piperazin-1-yl]-2-oxo-ethoxy}-phenyl)-methanesulfonic acid of formula IX is converted to the corresponding crystalline (5-chloro-2-{2-[4-(4-fluoro-benzyl)-2R,5S-dimethyl-piperazin-1-yl]-2-oxo-ethoxy}-phenyl)-methanesulfonic acid arginine salt of formula I by first reacting 1× with L-arginine or D-arginine in a polar protic solvent, such as methanol, for a time period of 30 minutes to 12 hours, preferably 1 hour. The resulting salt may then be slurried in a solvent, such as propanol or ethanol, at a temperature between 30° C. and 80° C., preferably about 45° C. The reaction is stirred for a time period of 1 to 4 days, preferably 2 days.

In reaction 7 the (5-chloro-2-{2-[4-(4-fluoro-benzyl)-2R,5S-dimethyl-piperazin-1-yl]-2-oxo-ethoxy}-phenyl)-methanesulfonic acid of formula IX is converted to the corresponding crystalline (5-chloro-2-{2-[4-(4-fluoro-benzyl)-2R,5S-dimethyl-piperazin-1-yl]-2-oxo-ethoxy}-phenyl)-methanesulfonic acid ethylene diamine salt of formula II by reacting 1× with ethylene diamine in an aprotic solvent, such as actetonitrile, at a temperature between 30° C. and 80° C., preferably about 45° C. The reaction is stirred for a time period of 1 to 5 days, preferably 3 days.

In reaction 8 the (5-chloro-2-{2-[4-(4-fluoro-benzyl)-2R,5S-dimethyl-piperazin-1-yl]-2-oxo-ethoxy}-phenyl)-methanesulfonic acid of formula IX is converted to the corresponding crystalline (5-chloro-2-{2-[4-(4-fluoro-benzyl)-2R,5S-dimethyl-piperazin-1-yl]-2-oxo-ethoxy}-phenyl)-methanesulfonic acid ethylene diamine salt of formula III by reacting 1× with calcium hydroxide in a polar protic solvent, such as water, for a time period of 1 to 5 days, preferably 3 days.

Unless indicated otherwise, the pressure of each of the above reactions is not critical. Generally, the reactions will be conducted at a pressure of about one to about three atmospheres, preferably at ambient pressure (about one atmosphere).

Compounds of the formula Ia and their pharmaceutically acceptable salts (hereinafter also referred to, collectively, as “the active compounds”) are potent inhibitors of MIP-1α (CCL3) binding to its receptor CCR1 found on inflammatory and immunomodulatory cells (preferably leukocytes and lymphocytes). The CCR1 receptor is also sometimes referred to as the CC-CKR1 receptor. These compounds also inhibit MIP-1α (and the related chemokines shown to interact with CCR1 (e.g., RANTES (CCL5), MCP-2 (CCL8), MCP-3 (CCL7), HCC-1 (CCL14) and HCC-2 (CCL15))) induced chemotaxis of THP-1 cells and human leukocytes and are potentially useful for the treatment and prevention of the following disorders and conditions: autoimmune diseases (such as rheumatoid arthritis, Takayasu arthritis, psoriatic arthritis, juvenile arthritis, ankylosing spondylitis, type I diabetes (recent onset), lupus, inflammatory bowel disease, Chrohn's disease, optic neuritis, psoriasis, neuroimmunologic disease (multiple sclerosis (MS) primary progressive MS, secondary progressive MS, chronic progressive MS, progressive relapsing MS, relapsing remitting MS, worsening MS), polymyalgia rheumatica, uveitis, thyroiditis and vasculitis); fibrosis (such as pulmonary fibrosis (for example idiopathic pulmonary fibrosis, interstitial pulmonary fibrosis), fibrosis associated with end-stage renal disease, fibrosis caused by radiation, tubulointerstitial fibrosis, subepithelial fibrosis, scleroderma (progressive systemic sclerosis), hepatic fibrosis (including that caused by alcoholic or viral hepatitis), primary and secondary biliary cirrhosis); allergic conditions (such as asthma, contact dermatitis and atopic dermatitis); acute and chronic inflammatory conditions including ocular inflammation, stenosis, lung inflammation (such as chronic bronchitis, chronic obstructive pulmonary disease, adult Respiratory Distress Syndrome, Respiratory Distress Syndrome of infancy, immune complex alveolitis), vascular inflammation resulting from tissue transplant or during restenosis (including, but not limited to, restenosis following angioplasty and/or stent insertion) and other acute and chronic inflammatory conditions (such as synovial inflammation caused by arthroscopy, hyperuremia, or trauma, osteoarthritis, ischemia reperfusion injury, glomerulonephritis, nasal polyosis, enteritis, Behcet's disease, preeclampsia, oral lichen planus, Guillian-Barre syndrome); acute and chronic transplant rejection (including xeno-transplantation); HIV infectivity (co-receptor usage); granulomatous diseases (including sarcoidosis, leprosy and tuberculosis); Alzheimer's disease; chronic fatigue syndrome; pain; atherosclerosis; conditions associated with leptin production (such as obesity, cachexia, anorexia, type II diabetes, hyperlipidemia and hypergonadism); and sequelae associated with certain cancers such as multiple myeloma. This method of treatment may also have utility for the prevention of cancer metastasis, including but not limited to breast cancer.

This method of treatment may also inhibit the production of metalloproteinases and cytokines at inflammatory sites (including but not limited to MMP9, TNF, IL-1, and IL-6) either directly or indirectly (as a consequence of decreasing cell infiltration) thus providing benefit for diseases or conditions linked to these cytokines (such as joint tissue damage, hyperplasia, pannus formation and bone resorption, hepatic failure, Kawasaki syndrome, myocardial infarction, acute liver failure, septic shock, congestive heart failure, pulmonary emphysema or dyspnea associated therewith). This method of treatment may also prevent tissue damage caused by inflammation induced by infectious agents (such as viral induced encephalomyelitis or demyelination, viral inflammation of the lung or liver (e.g. caused by influenza or hepatitis), gastrointestinal inflammation (for example, resulting from H. pylori infection), inflammation resulting from: bacterial meningitis, HIV-1, HIV-2, HIV-3, cytomegalovirus (CMV), adenoviruses, Herpes viruses (Herpes zoster and Herpes simplex) fungal meningitis, lyme disease, malaria).

The activity of the compounds of the invention can be assessed according to procedures know to those of ordinary skill in the art. Examples of recognized methods for determining CCR1 induced migration can be found in Coligan, J. E., Kruisbeek, A. M., Margulies, D. H., Shevach, E. M., Strober, W. editors: Current Protocols In Immunology, 6.12.1-6.12.3. (John Wiley and Sons, NY, 1991). One specific example of how to determine the activity of a compound for inhibiting migration is described in detail below.

### Chemotaxis Assay

The ability of compounds to inhibit the chemotaxis to various chemokines can be evaluated using standard 48 or 96 well Boyden Chambers with a 5 micron polycarbonate filter. All reagents and cells can be prepared in standard RPMI (BioWhitikker Inc.) tissue culture medium supplemented with 1 mg/ml of bovine serum albumin. Briefly, MIP-1α (Peprotech, Inc., P.O. Box 275, Rocky Hill N.J.) or other test agonists were placed into the lower chambers of the Boyden chamber. A polycarbonate filter was then applied and the upper chamber fastened. The amount of agonist chosen is that determined to give the maximal amount of chemotaxis in this system (e.g., 1 nM for MIP-1α should be adequate).

THP-1 cells (ATCC TIB-202), primary human monocytes, or primary lymphocytes, isolated by standard techniques can then be added to the upper chambers in triplicate together with various concentrations of the test compound. Compound dilutions can be prepared using standard serological techniques and are mixed with cells prior to adding to the chamber.

After a suitable incubation period at 37 degrees centigrade (e.g. 3.5 hours for THP-1 cells, 90 minutes for primary monocytes), the chamber is removed, the cells in the upper chamber aspirated, the upper part of the filter wiped and the number of cells migrating can be determined according to the following method.

For THP-1 cells, the chamber (a 96 well variety manufactured by Neuroprobe) can be centrifuged to push cells off the lower chamber and the number of cells can be quantitated against a standard curve by a color change of the dye fluorocein diacetate.

For primary human monocytes, or lymphocytes, the filter can be stained with Dif Quik® dye (American Scientific Products) and the number of cells migrating can be determined microscopically.

The number of cells migrating in the presence of the compound are divided by the number of cells migrating in control wells (without the compound). The quotant is the % inhibition for the compound which can then be plotted using standard graphics techniques against the concentration of compound used. The 50% inhibition point is then determined using a line fit analysis for all concentrations tested. The line fit for all data points must have an coefficient of correlation (R squared) of >90% to be considered a valid assay.

All of the compounds of the invention that were tested had IC₅₀ of less than 10 μM, in the Chemotaxis assay.

The compositions of the present invention may be formulated in a conventional manner using one or more pharmaceutically acceptable carriers. Thus, the active compounds of the invention may be formulated for oral, buccal, intranasal, topical, transdermal, parenteral (e.g., intravenous, intramuscular or subcutaneous) ocular or rectal administration or in a form suitable for administration by inhalation or insufflation. The active compounds of the invention may also be formulated for sustained delivery.

For oral administration, the pharmaceutical compositions may take the form of, for example, tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); and preservatives (e.g, methyl or propyl p-hydroxybenzoates or sorbic acid).

For buccal administration, the composition may take the form of tablets or lozenges formulated in conventional manner. Moreover, quick dissolve tablets may be formulated for sublingual absorption.

The active compounds of the invention may be formulated for parenteral administration by injection, including using conventional catheterization techniques or infusion. Formulations for injection may be presented in unit dosage form, e.g., in ampules or in multi-dose containers, with an added preservative. The compositions may take such forms as suspensions, solutions or emulsions in oily or aqueous vehicles, and may contain formulating agents such as suspending, stabilizing and/or dispersing agents. Alternatively, the active ingredient may be in powder form for reconstitution with a suitable vehicle, e.g., sterile pyrogen-free water, before use.

The active compounds of the invention may also be formulated in rectal compositions such as suppositories or retention enemas, e.g., containing conventional suppository bases such as cocoa butter or other glycerides.

For intranasal administration or administration by inhalation, the active compounds of the invention are conveniently delivered in the form of a solution or suspension from a pump spray container that is squeezed or pumped by the patient or as an aerosol spray presentation from a pressurized container or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. The pressurized container or nebulizer may contain a solution or suspension of the active compound. Capsules and cartridges (made, for example, from gelatin) for use in an inhaler or insufflator may be formulated containing a powder mix of a compound of the invention and a suitable powder base such as lactose or starch to provide for dry powder inhalation.

A proposed dose of the active compounds of the invention for oral, parenteral, nasal, or buccal administration to the average adult human for the treatment of the conditions referred to above (e.g., rheumatoid arthritis) is 0.1 to 1000 mg of the active ingredient per unit dose which could be administered, for example, 1 to 4 times per day.

Aerosol formulations for treatment of the conditions referred to above (e.g., rheumatoid arthritis) in the average adult human are preferably arranged so that each metered dose or “puff” of aerosol contains 20 μg to 1000 μg of the compound of the invention. The overall daily dose with an aerosol will be within the range 0.1 mg to 1000 mg. Administration may be several times daily, for example 2, 3, 4 or 8 times, giving for example, 1, 2 or 3 doses each time.

The active agents may be formulated for sustained delivery according to methods well known to those of ordinary skill in the art. Examples of such formulations can be found in U.S. Pat. Nos. 3,538,214, 4,060,598, 4,173,626, 3,119,742, and 3,492,397, all of which are incorporated herein in their entireties for all purposes.

The compounds of the invention may also be utilized in combination therapy with other therapeutic agents such as those that inhibit immune cell activation and/or cytokine secretion or action (i.e. Cyclosporin A, ISAtx247, Rapamycin, Everolimus, FK-506, Azathioprine, Mycophenolate motetil, Mycophenolic acid, Daclizumab, Basiliximab, Muromonab, Horse anti-thymocyte globulin, Polyclonal rabbit antithymocyte globulin, Leflunomide, FK-778 (MNA-715), FTY-720, BMS-188667 (CTLA4-1 g), BMS-224818 (CTLA4-1 g), RG-1046<CTLA4-1 g), Prednisone, Prednisolone, Methylprednisolone suleptanate, Cortisone, Hydrocortisone, Methotrexate, Sulfasalazine, Etanercept, Infliximab, Adalimumab (D2E7), CDP-571, CDP-870, Anakinra, Anti-interleukin-6 receptor monoclonal antibody (MRA)), NSAIDS (aspirin, acetaminophen, naproxen, ibuprofen, ketoprofen, diclofenac and piroxicam), COX-2 inhibitors (Celecoxib, Valdecoxib, Rofecoxib, Parecoxib, Etoricoxib, L-745337, COX-189, BMS-347070, S-2474, JTE-522, CS-502, P-54, DFP), Glatiramer acetate, Interferon beta 1-a, Interferon beta 1-b, Mitoxantrone, Pimecrolimus, or agents that inhibit cell recruitment mechanisms (eg inhibitors of integrin upregulation or function) or alter leukocyte trafficking.

### EXPERIMENTAL

The following examples are put forth so as to provide those of ordinary skill in the art with a complete disclosure and description of how the compounds, compositions, and methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.) but some errors and deviations should be accounted for. Unless indicated otherwise, percent is percent by weight given the component and the total weight of the composition, temperature is in ° C. or is at ambient temperature, and pressure is at or near atmospheric. Commercial reagents were utilized without further purification.

Note that all numbers provided herein are approximate, but effort have been made to ensure accuracy with respect to numbers (e.g., amounts, temperature, etc.); however some errors and deviations should be accounted for.

### Example 1

### (5-Chloro-2-{2-[4-(4-fluoro-benzyl)-2R,5S-dimethyl-piperazin-1-yl]-2-oxo-ethoxy}-phenyl)-methanesulfonic acid Arginine Salt, form A

2-Chloro-1-[4-(4-fluoro-benzyl)-2R,5S-dimethyl-piperazin-1-yl]-ethanone

1-(4-Fluoro-benzyl)-2R,5S-dimethyl-piperazine (1 equiv) and triethylamine (1.1 equiv) were dissolved in toluene at 10° C. Chloroacetyl chloride (1.1 equiv) was introduced dropwise and the reaction maintained at −10° C. until the acylation was complete (about 1 hour). At this time the reaction was quenched by addition of water. The layers were vigorously mixed for 5 minutes and then allowed to separate, and the aqueous layer was withdrawn. The organic layer was washed with one additional portion of water, the aqueous layer was withdrawn, and the combined aqueous layers were extracted once with toluene. The organic layers were combined and dried by azeotropic distillation at atmospheric pressure, monitoring the head temperature until it stabilized at 110-111° C. for about 5 minutes. This dark solution was then cooled to an internal temperature of 85° C. and used directly in the next reaction.

5-Chloro-2-{2-[4-(4-fluoro-benzyl)-2R,5S-dimethyl-piperazin-1-yl]-2-oxo ethoxy}-benzaldehyde

The toluene solution of 2-chloro-1-[4-(4-fluoro-benzyl)-2R,5S-dimethyl-piperazin-1-yl]-ethanone prepared in the last step was maintained at 85° C. as 5-chlorosalicylaldehyde (1.1 equiv) and potassium carbonate (1.5 equiv) were introduced in single portions as solids, followed by the addition of 10 vol % of DMAc. After 12-13 hours the dark reaction mixture was cooled to ambient temperature and diluted with water. The two layers were mixed for 10 minutes and then allowed to separate, and the yellow, cloudy lower aqueous layer was removed. The red organic layer was retained and washed with a 1 N sodium hydroxide solution and the aqueous layer removed. The organic layer was washed once more with water, and after agitating 10 min the resulting mild emulsion was allowed to stand for 1 hour to break. The organic layer was removed, dried with sodium sulfate and concentrated to a yellow solid. 5-Chloro-2-{2-[4-(4-fluoro-benzyl)-(2R,5S)-2,5-dimethyl-piperazin-1-yl]-2-oxo-ethoxy}-benzaldehyde was isolated (94% yield over two steps).

2-(4-Chloro-2-hydroxymethyl-phenoxy)-1-[4-(4-fluoro-benzyl)-2R,5S-dimethyl-piperazin-1-yl]-ethanone

5-Chloro-2-{2-[4-(4-fluoro-benzyl)-2R,5S-dimethyl-piperazin-1-yl]-2-oxo-ethoxy}-benzaldehyde (1 equiv) was dissolved in a 4:1 mixture of EtOH:THF and cooled to 0-10° C. NaBH₄ pellets (1.2H equiv) were introduced in one portion, and the reaction mixture was stirred at 0-10° C. for one hour and then allowed to warm to ambient temperature. After an additional hour the reaction was quenched by the addition of 1 N NaOH and toluene. The solution was concentrated under reduced pressure to remove the EtOH, and the residue diluted with toluene and washed with water. The organic layer was concentrated under reduced pressure to an oil that was dissolved in CH₂Cl₂ and concentrated on silica gel. This material was loaded on a silica gel column (packed with hexanes) and the title compound was eluted as the most polar UV-active compound using a gradient of hexanes to 2:1 hexanes:EtOAc. After decolorization of the non-colorless fractions with Darco, the title compound was isolated as a pale yellow foam (89% yield).

2-(4-Chloro-2-chloromethyl-phenoxy)-1-[4-(4-fluoro-benzyl)-2R,5S-dimethyl-piperazin-1-yl]-ethanone

2-(4-Chloro-2-hydroxymethyl-phenoxy)-1-[4-(4-fluoro-benzyl)-2R,5S-dimethyl-piperazin-1-yl]-ethanone (1 equiv) was dissolved in CH₂Cl₂ and DMF was added. Thionyl chloride (1.1 equiv) was introduced dropwise over approximately one hour. Upon completion of this addition the reaction mixture was quenched with water, and the two layers were separated. The organic layer was vigorously mixed with saturated NaHCO₃ to bring the pH to 7-8. The layers were again separated, and the organic layer was dried with Na₂SO₄ and concentrated under reduced pressure to give title compound (99% yield), which was carried on to the next step without further purification.

(5-Chloro-2-{2-[4-(4-fluoro-benzyl)-2R,5S-dimethyl-piperazin-1-yl]-2-oxo-ethoxy}-phenyl)-methanesulfonic acid Sodium Salt

2-(4-Chloro-2-chloromethyl-phenoxy)-1-[4-(4-fluoro-benzyl)-2R,5S-dimethyl-piperazin-1-yl]-ethanone (1 equiv) was dissolved in acetonitrile and added to a solution of Na₂SO₃ (3 equiv) in water. The resulting clear solution was heated to reflux for 6 hours, at which time the reaction mixture was then cooled to ambient temperature and filtered to remove precipitated Na₂SO₃. The phases were separated and the organic layer was washed once with brine. The combined aqueous layers were extracted once with acetonitrile and the combined organic layers concentrated under reduced pressure. This yellow oil, which consisted of a mixture of (5-chloro-2-{2-[4-(4-fluoro-benzyl)-2R,5S-dimethyl-piperazin-1-yl]-2-oxo-ethoxy}-phenyl)-methanesulfonic acid sodium salt and benzyl alcohol in a ratio of 89:7, was extracted with isopropyl ether to purge alcohol and then concentrated under reduced pressure to provide a light yellow foam that still contained about 2% of benzyl alcohol as the primary contaminant.

Additional purification of this material was conducted as follows. (5-Chloro-2-{2-[4-(4-fluoro-benzyl)-2R,5S-dimethyl-piperazin-1-yl]-2-oxo-ethoxy}-phenyl)-methanesulfonic acid sodium salt was dissolved in CH₂Cl₂ and clarified by filtration through a celite pad. The clear filtrate was then heated to 45° C. and diluted by portionwise additions of isopropyl ether until white solids formed and did not dissolve on continued heating. The internal temperature of the reaction was then cooled to 25° C. over 4 hours and stirring was continued at this temperature for an additional 12 hours. At this time the solids were isolated and washed with 1 L isopropyl ether and then dried at 40° C./3 mmHg to give the title compound as a 98.8:0.37 ratio of desired product to benzyl alcohol. The mother liquor was re-filtered to isolate the solids that precipitated during the initial filtration; this provided an additional amount of title compound at a purity level of 98.4% (0.52% benzyl alchol). The combined yield for this reaction was 80%.

(5-Chloro-2-{2-[4-(4-fluoro-benzyl)-2R,5S-dimethyl-piperazin-1-yl]-2-oxo-ethoxy}-phenyl)-methanesulfonic acid

(5-Chloro-2-{2-[4-(4-fluoro-benzyl)-2R,5S-dimethyl-piperazin-1-yl]-2-oxo-ethoxy}-phenyl)-methanesulfonic acid sodium salt (1 equiv) was dissolved over several hours in acetonitrile. Concentrated HCl (0.96 equiv) and then 1H HCl (0.04 equiv) were added slowly to the solution, which resulted in immediate precipitation of (5-Chloro-2-{2-[4-(4-fluoro-benzyl)-2R,5S-dimethyl-piperazin-1-yl]-2-oxo-ethoxy}-phenyl)-methanesulfonic acid as a thick gum on the bottom of the flask. Using efficient overhead stirring, the solution was warmed to 50° C. for 12 hours and then brought to ref lux, which partially dissolved the residue. One additional aliquot of 1 N HCl (0.08 equiv) was added slowly to adjust the pH of the solution to −3. Solvent was distilled off in order to remove the water by azeotrope, and the solution was recharged with anhydrous acetonitrile. The solution was brought to reflux again and cooled slowly to 60° C. Stirring was stopped, allowing solids to settle to the bottom of the flask, and the light orange solution was removed. The flask was charged with acetonitrile and stirring was resumed. After bringing this solution to reflux, it was cooled slowly to 60° C. before the acetonitrile layer was again removed. This procedure was repeated once more; the acetonitrile layer was isolated by filtration through a plug of celite prepared with hot acetonitrile to remove precipitated NaCl. The filtrates were concentrated to give white/tan solids (96% yield).

(5-Chloro-2-{2-[4-(4-fluoro-benzyl)-2R,5S-dimethyl-piperazin-1-yl]-2-oxo-ethoxy}-phenyl)-methanesulfonic acid Arginine Salt, form A

(5-Chloro-2-{2-[4-(4-fluoro-benzyl)-2R,5S-dimethyl-piperazin-1-yl]-2-oxo-ethoxy}-phenyl)-methanesulfonic acid (1.0 equiv) was dissolved in methanol and heated to 45° C. under nitrogen at atmospheric pressure. L-Arginine (1.0 equiv) was added as a solid to the orange solution. Most of the arginine dissolved within 30 minutes of stirring, and water was added to help solubilize the rest. The pressure was reduced to 300 mmHg and methanol was removed by distillation at a pot temperature of 42-45° C., during which time some crystallization occurred in the reaction flask. The distilled solvent was replaced in the reaction flask with n-propanol, and this process was repeated twice more to exchange all the methanol for n-propanol. The resulting white slurry was then stirred at 45° C. for 48 hours. At this time the white solids were isolated by filtration, were washed with n-propanol, and dried under reduced pressure at 40° C. to constant mass (90% yield).

Alternatively, this last step may be carried out using the following procedure. (5-Chloro-2-{2-[4-(4-fluoro-benzyl)-2R,5S-dimethyl-piperazin-1-yl]-2-oxo-ethoxy}-phenyl)-methanesulfonic acid (1.0 equiv) was dissolved in ethyl alcohol (3 parts) and acetonitrile (2 parts) and heated to 70 to 80° C. over a period of 15 to 25 min. L-Arginine (about 1.0 equiv) dissolved in water was added to the ethanol/acetonitrile solution. The pressure was reduced and the compound was concentrated by distillation at a pot temperature of 78 to 82° C., during which time ethyl alcohol was added to the reaction flask. The resulting material was then cooled to a temperature of 23 to 27° C. and held for about 0.8 to 1.2 hr. At which time, the material was heated to a temperature of 40 to 50° C. and held for 16 to 20 hours. Subsequently, the material was cooled to a temperature of 20 to 30° C. and held for 2 to 6 hours. At this time the material was isolated by filtration, washed with ethyl alcohol, and dried under reduced pressure at 40 to 50° C. to constant mass (about 80% yield).

### Example 2

### (5-Chloro-2-{2-[4-(4-fluoro-benzyl)-2R,5S-dimethyl-piperazin-1-yl]-2-oxo-ethoxy}-phenyl)-methanesulfonic Acid Ethylene Diamine Salt

(5-Chloro-2-{2-[4-(4-fluoro-benzyl)-2R,5S-dimethyl-piperazin-1-yl]-2-oxo-ethoxy}-phenyl)-methanesulfonic acid (1.0 equiv.) was dissolved in acetonitrile and warmed to 45° C. Ethylene diamine (0.5 equiv.) was added and the solution stirred for 72 hours. The resulting suspension was filtered and the salt washed with cold acetonitrile. The title compound was dried under reduced pressure at 45° C. to constant mass (82% yield).

### Example 3

### (5-Chloro-2-{2-[4-(4-fluoro-benzyl)-2R,5S-dimethyl-piperazin-1-yl]-2-oxo-ethoxy}-phenyl)-methanesulfonic Acid Calcium Salt

(5-Chloro-2-{2-[4-(4-fluoro-benzyl)-2R,5S-dimethyl-piperazin-1-yl]-2-oxo-ethoxy}-phenyl)-methanesulfonic acid (1.0 equiv.) was dissolved in 80° C. water. Calcium hydroxide (0.5 equiv.) was added and the mixture was allowed to cool to ambient temperature. After 72 hours the suspension was filtered and the salt washed with cold water and then diethyl ether. The title compound was dried under reduced pressure at 45° C. to constant mass (84% yield).

### Example 4

### (5-Chloro-2-{2-[4-(4-fluoro-benzyl)-2R,5S-dimethyl-piperazin-1-yl]-2-oxo-ethoxy}-phenyl)-methanesulfonic Acid Arginine Salt, form B

Form B was isolated from slurries of form A in methanol, acetonitrile, and chloroform at ambient temperature and slurries of form A in acetonitrile and chloroform at elevated temperature. It contains 1-10% of the crystallization solvent. Form B was also obtained when amorphous material was slurried in acetonitrile at elevated temperature.

Form B appeared as agglomerated flakes by polarized light microscopy.

### Example 5

### (5-Chloro-2-{2-[4-(4-fluoro-benzyl)-2R,5S-dimethyl-piperazin-1-yl]-2-oxo-ethoxy}-phenyl)-methanesulfonic Acid Arginine Salt, Form C

Form C crystals were prepared by slurrying Form A in methylenechloride by evaporation. It is an anhydrate containing less than 1% organic solvent. Form C appeared as large fused flakes by PLM.

### Example 6

### (5-Chloro-2-{2-[4-(4-fluoro-benzyl)-2R,5S-dimethyl-piperazin-1-yl]-2-oxo-ethoxy}-phenylmethanesulfonic Acid Arginine Salt, Form E

Form E was an anhydrate and obtained from methylenechloride by slurry amorphous material at 25 to 50° C. It appears as large or fused flakes by PLM.

### Example 7

### (5-Chloro-2-{2-[4-(4-fluoro-benzyl)-2R,5S-dimethyl-piperazin-1-yl]-2-oxo-ethoxyy}-phenyl)-methanesulfonic Acid Arginine Salt, Amorphous

(5-Chloro-2-{2-[4-(4-fluoro-benzyl)-2R,5S-dimethyl-piperazin-1-yl]-2-oxo-ethoxy}-phenyl)-methanesulfonic acid arginine salt was slurried with water and organic/water mixtures and methanol. Bulk amorphous material was obtained by rotary vacuum evaporating a viscous aqueous solution to dryness.

Throughout this application, various publications are referenced. The disclosures of these publications in their entireties are hereby incorporated by reference into this application for all purposes.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A compound of (5-chloro-2-{2-[4-(4-fluoro-benzyl)-2R,5S-dimethyl-piperazin-1-yl]-2-oxo-ethoxy}-phenyl)-methanesulfonic acid or an arginine, ethylene diamine or calcium salt thereof, having an amorphous or crystalline form.

2. The compound according to claim 1, wherein the compound is the amorphous form of the (5-chloro-2-{2-[4-(4-fluoro-benzyl)-2 R,5S-dimethyl-piperazin-1-yl]-2-oxo-ethoxy}-phenyl)-methanesulfonic acid arginine salt.

3. The compound according to claim 1, wherein the compound is the crystalline form of the (5-chloro-2-{2-[4-(4-fluoro-benzyl)-2 R,5S-dimethyl-piperazin-1-yl]-2-oxo-ethoxy}-phenyl)-methanesulfonic acid arginine salt having a powder X-ray diffraction pattern comprising high intensity peaks expressed in degrees two-theta at approximately 4.1, 12.5, 16.7, 18.4, 19.5, 20.1, 20.9 and 24.0.

4. The compound according to claim 1, wherein the compound is the crystalline form of the (5-chloro-2-{2-[4-(4-fluoro-benzyl)-2R,5S-dimethyl-piperazin-1-yl]-2-oxo-ethoxy}-phenyl)-methanesulfonic acid arginine salt having a powder X-ray diffraction pattern comprising high intensity peaks expressed in degrees two-theta at approximately 4.1, 10.8, 11.4, 12.2, 12.5, 12.9, 13.2, 13.7, 14.6, 15.4, 16.1, 16.7, 17.8, 18.2, 18.4, 19.5, 20.1, 20.9, 21.3, 21.8, 22.8, 24.0, 25.1, 25.7, 26.8, 27.1, 28.2, 29.0, 29.5, 30.6, 31.0, and 32.3.

5. The compound according to claim 1, wherein the compound is the crystalline form of the (5-chloro-2-{2-[4-(4-fluoro-benzyl)-2 R,5S-dimethyl-piperazin-1-yl]-2-oxo-ethoxy}-phenyl)-methanesulfonic acid arginine salt having a powder X-ray diffraction pattern comprising peaks expressed in degree two-theta at approximately 4.0, 11.1, 16.0, 17.3, 17.5, 18.2, 18.4, 19.2, 19.6, 20.0, 21.6, and 22.2.

6. The compound according to claim 1, wherein the compound is the crystalline form of the (5-chloro-2-{2-[4-(4-fluoro-benzyl)-2R,5S-dimethyl-piperazin-1-yl]-2-oxo-ethoxy}-phenyl)-methanesulfonic acid arginine salt having a powder X-ray diffraction pattern comprising peaks expressed in degree two-theta at approximately 4.1, 20.5, and 24.7.

7. The compound according to claim 1, wherein the compound is the crystalline form of the (5-chloro-2-{2-[4-(4-fluoro-benzyl)-2R,5S-dimethyl-piperazin-1-yl]-2-oxo-ethoxy}-phenyl)-methanesulfonic acid arginine salt having a powder X-ray diffraction pattern comprising peaks expressed in degree two-theta at approximately 3.7, 7.3, 11.0, 18.3, 19.7, 22.1, 22.9, and 25.8.

8. The compound according to any one of claims 3-4, wherein the compound is the crystalline form of the (5-chloro-2-{2-[4-(4-fluoro-benzyl)-2R,5S-dimethyl-piperazin-1-yl]-2-oxo-ethoxy}-phenyl)-methanesulfonic acid arginine salt having a differential scanning calorimetry thermogram comprising an endothermic event with an onset temperature of approximately 200° C. using a heating rate of about 5° C. per minute from about 30° C. to about 300° C.

9. The compound according to any one of claims 3-4, wherein the compound is the crystalline form of the (5-chloro-2-{2-[4-(4-fluoro-benzyl)-2R,5S-dimethyl-piperazin-1-yl]-2-oxo-ethoxy}-phenyl)-methanesulfonic acid arginine salt having a solid state nuclear magnetic resonance spectrum comprising ¹³C chemical shifts expressed in parts per million at approximately 174.9, 174.1, 167.3, 166.4, 163.3, 162.8, 161.4, 160.8, 158.8, 157.2, 154.4, 134.3, 133.7, 132.3, 131.5, 130.8, 128.1, 125.8, 124.7, 123.6, 117.7, 116.8, 114.7, 111.7, 72.6, 67.0, 58.1, 56.0, 55.1, 52.8, 51.9, 51.0, 49.0, 46.8, 43.0, 41.3, 27.7, 26.3, 24.8, 23.3, 17.9, 15.4, 9.5, and 7.4.

10. A pharmaceutical composition comprising an amount of a compound of claim 1 and a pharmaceutically acceptable carrier.

11. A method for treating or preventing a disorder or condition in a subject that can be treated or prevented by antagonizing the CCR1 receptor or inhibiting the production of metalloproteinase or cytokine at an inflammatory site comprising the step of administering to the subject an effective amount of the compound of claim 1.

12. The method according to claim 11, wherein said disorder or condition is selected from the group consisting of autoimmune diseases, acute and chronic inflammatory conditions, allergic conditions, infection associated with inflammation, viral inflammation, transplantation tissue rejection, atherosclerosis, restenosis, HIV infectivity, granulomatous diseases in a mammal, fibrosis, Alzheimer's disease, conditions associated with leptin production, sequelae associated with cancer, cancer metastasis, diseases or conditions related to production of cytokines at inflammatory sites, and tissue damage caused by inflammation induced by infectious agents.

13. The method according to claim 12, wherein said disorder or condition is rheumatoid arthritis, Takayasu arthritis, psoriatic arthritis, ankylosing spondylitis, type I diabetes (recent onset), lupus, inflammatory bowel disease, Chrohn's disease, optic neuritis, psoriasis, multiple sclerosis, polymyalgia rheumatica, uveitis, thyroiditis and vasculitis, pulmonary fibrosis, fibrosis associated with end-stage renal disease, fibrosis caused by radiation, tubulointerstitial fibrosis, subepithelial fibrosis, scleroderma, hepatic fibrosis, primary and secondary biliary cirrhosis, asthma, contact dermatitis, atopic dermatitis, chronic bronchitis, chronic obstructive pulmonary disease, adult Respiratory Distress Syndrome, Respiratory Distress Syndrome of infancy, immune complex alveolitis, synovial inflammation caused by arthroscopy, hyperuremia, osteoarthritis, ischemia reperfusion injury, glomerulonephritis, nasal polyosis, enteritis, Behcet's disease, preeclampsia, oral lichen planus, Guillian-Barre syndrome, sarcoidosis, leprosy, tuberculosis, obesity, cachexia, anorexia, type II diabetes, hyperlipidemia and hypergonadism, sequelae associated with multiple myeloma, breast cancer, joint tissue damage, hyperplasia, pannus formation and bone resorption, hepatic failure, Kawasaki syndrome, myocardial infarction, acute liver failure, septic shock, congestive heart failure, pulmonary emphysema or dyspnea associated therewith, viral induced encephalomyelitis or demyelination, viral inflammation of the lung or liver, gastrointestinal inflammation, bacterial meningitis, HIV-1, HIV-2, HIV-3, cytomegalovirus, adenoviruses, Herpes viruses, fungal meningitis, lyme disease, or malaria.
